(19) United States
(12) Patent Application Publication
(10) Pub. No.: US 2007/0015793 A1
(43) Pub. Date: Jan. 18, 2007

(54) Synthesis of nojirimycins

(75) Inventors: Bradford H. Hirth, Littleton, MA (US); Glen Rennie, Somerville, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: MACROZYME DNM B.V., Amersterdam (NL)

(21) Appl. No.: 11/019,659

(22) Filed: Dec. 22, 2004

Related U.S. Application Data
(60) Provisional application No. 60/532,153, filed on Dec. 23, 2003.

(51) Int. Cl. A61K 31/445

(52) U.S. Cl. 514/327; 546/242

(57) ABSTRACT

Disclosed are methods of preparing substituted nojirimycins represented by the following formula, 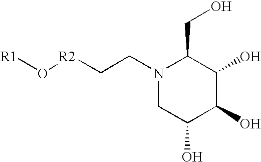
comprising reacting 1-deoxynojirimycin with a reagent prepared by reacting an oxidizing agent with the compound represented by the following formula.

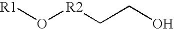

### RELATED APPLICATION

This application claims the benefit of U.S. Provisional Application No. 60/532,153, filed Dec. 23, 2003, the entire teachings of which is incorporated herein by reference.

### BACKGROUND OF THE INVENTION

Ceramide signalling processes are involved or implicated in a wide variety of diseases, including Gaucher's disease. Current treatment methods for Gaucher's disease are among the most expensive treatments for a single disease, exceeding in some cases $400,000 USD per patient per year. It has recently been disclosed in WO 98/02161, the entire teachings of which are incorporated herein by reference, that enzymes associated with ceramide signalling, such as glucosylceramidase and glucocerebreosidase, can be inhibited with derivatives of nojirimycin such Compound 1: 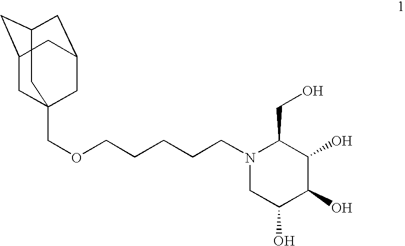
Such compounds can be used to treat diseases associated with ceramide signalling, for example, Gaucher's disease.

The key step in preparing these compounds is reductively aminating an aldehyde intermediate, for example the aldehyde in Compound 2: 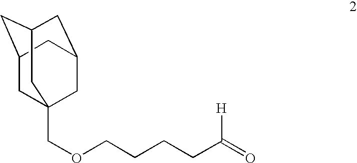
Unfortunately, synthetic routes towards these substituted aldehyde intermediates in the prior art result in a low overall yield of 20% or less, leading to overall yields of compounds such as represented by structural formula 1, of 10% or less, as disclosed in WO 98/02161. There is therefore a need for new synthetic methods that efficiently produce pharmacologically active nojirimycin derivatives such as Compound 1.

### SUMMARY OF THE INVENTION

It has now been found that substituted aldehyde intermediates such as Compound 2 can be prepared by an improved route, leading to improved overall synthesis of substituted nojirimycin derivatives such as Compound 1. The surprising and significant effect of this new route is that the overall yield is substantially increased over the prior art, and furthermore, the steps in this new route are more readily adapted to large-scale pharmaceutical production. Typically, overall yields of aldehyde intermediates such as Compound 2 starting from commercially available materials are 55% or greater over three steps (see Examples 4-6), compared to 20% in four steps disclosed in WO 98/02161. Overall yields of substituted nojirimycins such as Compound 1 starting from commercially available materials are 40% or greater over four steps (see Examples 1 and 4-6), compared to 10% in five steps disclosed in WO 98/02161.

The invention includes a method of preparing an alkene represented by structural formula I: 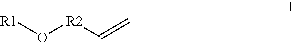
The method includes reacting an alcohol represented by R1-OH with an alkene represented by Y—R2—CH═CH₂, wherein R1 is an optionally substituted aromatic or aliphatic group, R2 is an aliphatic linking group, and Y is a leaving group.

Also included in the present invention is a method of preparing an alcohol represented by structural formula II: 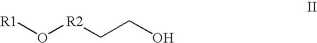
by reacting an alkene represented by structural formula I with a hydroboration reagent, thereby producing a hydroborated intermediate, followed by oxidation of the intermediate to form alcohol II, wherein R1 and R2 are as defined for structural formula I.

In another embodiment, the invention is a method of preparing an aldehyde represented by structural formula III: 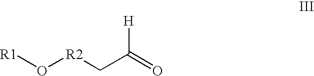
by reacting the alcohol represented by structural formula I with a oxidation reagent, wherein R1 and R2 are as defined for structural formula I.

In yet another embodiment, the invention is a method of preparing an N-alkylated nojirimycin derivative represented by structural formula IV: 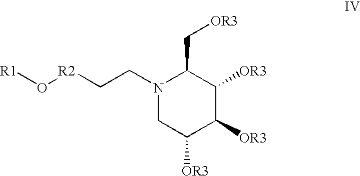
by reductively aminating the aldehyde represented by structural formula II with a 1-deoxynojirimycin derivative, such as that represented by structural formula V: 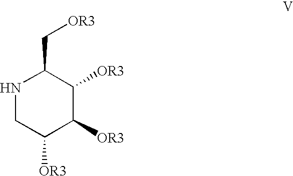
wherein R3 is —H or an alcohol protecting group, and R1 and R2 are as defined for structural formula I. In a preferred embodiment, R3 is —H, as in compound 1.

Another embodiment of the present invention is a compound represented by structural formula VI: 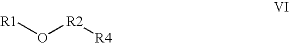
wherein R2 is an aliphatic linking group, R4 is —CH═CH₂ or —CH₂CH₂OH, and R1 is a group represented by one of structural formulas i-v:

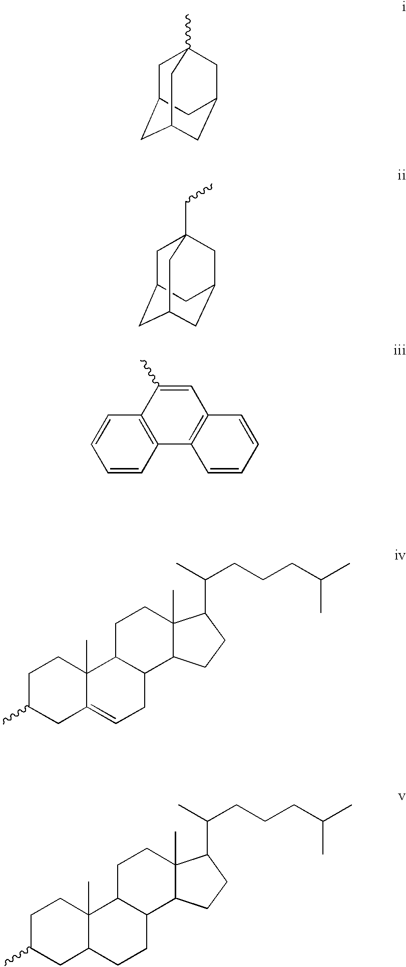

Thus, the present invention is also a method of preparing a 1-deoxynojirimycin derivative represented by structural formula IV from readily available starting materials R1-OH and Y—R2-CH═CH₂ by sequentially combining the reactions described above.

The advantages of the invention disclosed herein are significant. The improvements in the yield of the key intermediate allow pharmacologically active nojirimycins, including the glucosylceramidase inhibitors disclosed in WO 98/02161, to be made economically in pharmaceutically useful quantities. Furthermore, because this key intermediate is easily varied by appropriate choice of starting materials, it enables the preparation of a wide range of structural variants that can be used in screening assays for other therapeutic targets. Finally, the higher yield and concomitant lack of byproduct formation leads to less waste, and thus an environmentally responsible process.

### DETAILED DESCRIPTION OF THE INVENTION

The methods disclosed herein can be used to prepare derivatives of cyclic amines, such as N-alkylated nojirimycin derivatives represented by structural formula IV, and, in particular, Compound 1. The method includes sequentially preparing the compounds represented by structural formulas II to IV from the starting material represented by structural formula I using the reactions disclosed herein.

The alkene represented by structural formula I is prepared by alkylating a compound represented by structural formula R1—OH under basic conditions with a compound represented by structural formula Y—R2—CH═CH₂, where Y is a leaving group. A leaving group is a group that is displaced from a carbon atom upon attack by a nucleophile, e.g., under basic conditions, the R1—O⁻ anion acts as a nucleophile to alkylate Y—R2—CH═CH₂, displacing Y⁻. Such alkylation reactions and leaving groups are well-known to the art; see, for example, Larock, R C “Comprehensive Organic Transformations”, 2^{nd} ed., Wiley, New York, 1999, pp 890-893, and references cited therein, the entire teachings of which are incorporated herein by reference. Suitable leaving groups include, for example, a halogen or an optionally substituted sulfonate group. Suitable sulfonates include —OSO₂CH₃, —OSO₂CF₃, —OSO₂(4-methyphenyl), —OSO₂(4-bromophenyl), or —OSO₂(4-nitrophenyl). Alternatively, Y is —Cl, —Br, —I, —OSO₂CH₃, —OSO₂CF₃, or —OSO₂(4-methyphenyl). Preferably, Y is —Br.

In the alkylation reaction, alkene Y—R2—CH═CH₂ is typically used in a molar ratio relative to R1—OH of between about 1 and about 4, alternatively in a molar ratio of about 1.5 to about 3, and preferably about 2. The solvent used is a polar aprotic solvent or an ethereal solvent, preferably dimethyl sulfoxide. The base is an alkali hydroxide or alkoxide, for example, potassium hydroxide, in a molar ratio of about 2 to about 10, alternatively about 2 to about 6, and preferably about 4 relative to R1—OH. The reaction can be run between ambient temperature and 100° C., more preferably between 50°-100° C., or most preferably about 70° C. The reagents can be added in any order or simultaneously, preferably simultaneously. Representative conditions are provided in Example 6 in the Exemplification.

The alcohol represented by structural formula II is prepared by hydroborating a terminal alkene represented by structural formula I followed by oxidation of the intermediate to form II. Hydroboration methods are well known in the art; see, for example, “Hydroboration”, H. C. Brown, W. A. Benjamin, New York 1962; Larock, pp 1005-1009; and references cited therein, the entire teachings of which are incorporated herein by reference. A hydroboration reagent includes, for example, BH₃, B₂H₆, bis(3-methyl-2-butyl)borane, BH₂Cl, 9-borobicyclo[3.3.1]nonane (9-BBN), and the like. The hydroboration reagent is used in a molar ratio relative to alkene I of between about 1 to about 10, alternatively about 1 to about 5, and more preferably about 1 to about 3. Preferably, the hydroboration reagent is 9-BBN in a molar ratio of about 1.5. Typically, the subsequent oxidation step includes an excess of oxidation reagent selected from atmospheric oxygen or a peroxide, e.g., hydrogen peroxide, in combination with a base, e.g., an alkali metal hydroxide or alkoxide. Preferably, the oxidation step following hydroboration is conducted using sodium hydroxide and 30% hydrogen peroxide in a molar ratio of between about 5 to about 6 relative to I.

The hydroboration reagent and the alkene can be added simultaneously or in any order. Preferably, the hydroboration reagent is added to the alkene. These reagents are combined and allowed to react before adding the base and oxidation reagents. Suitable solvents ethereal, aromatic, and halogenated solvents, preferably ethereal solvents, most preferably diethyl ether, tetrahydrofuran, or a mixture of the two. Suitable reaction temperatures for each addition portion of the reaction, i.e., combining the alkene and the hydroboration reagent, or adding the oxidation reagent to the hydroborated intermediate, are in a range of between about −30° C. to about ambient temperature, more preferably, between −10° to 10° C., or most preferably, about 0° C. Representative conditions are provided in Example 5 in the Exemplification.

The aldehyde represented by structural formula III is prepared by oxidation of an alcohol represented by structural formula II. Suitable oxidation conditions for converting an alcohol to an aldehyde are well-known to the art, e.g., Larock, p 1235-1247, and references cited therein. Suitable oxidation conditions include electrolytic oxidation or an oxidation reagent, for example, potassium permanganate, pyridine/CrO₃, pyridinium chlorochromate, potassium dichromate, sodium dichromate, oxalyl chloride/dimethyl sulfoxide, and the like. An oxidation reagent can be used in an oxidative equivalent molar ratio relative to the alcohol represented by structural formula II of between about 1 and about 20, preferably between about 3 and about 10, and more preferably between about 3 to about 5. In a preferred embodiment, the oxidizing agent is a mixture of oxalyl chloride in a molar ratio of about 2.2 and dimethyl sulfoxide in a molar ratio of about 3.4 relative to the alcohol represented by structural formula II. The reagents can be combined in any order, simultaneously, or the DMSO can be added to the oxalyl chloride, followed by the alcohol represented by structural formula II. Preferably, the DMSO is added to the oxalyl chloride, followed by the alcohol represented by structural formula II. Suitable oxidation solvents are those that are not oxidized by the reaction, e.g., ethereal, aromatic, acidic, and halogenated solvents, and the like, preferably a halogenated solvent such as methylene chloride. Suitable reaction temperatures for the reaction are below ambient temperature, for example, between −78° to 10° C., typically −78° to −30° C., and preferably about −70° C. Representative conditions are provided in Example 4 in the Exemplification.

The last step in the disclosed method is the reductive amination of a substituted aldehyde such as III with an amine such as nojirimycin V. The disclosed method gives the product in 74% yield compared to only 50% yield disclosed in WO 98/02161.

For example, in a reductive amination reaction, the aldehyde represented by structural formula III is combined with nojirimycin, represented by structural formula V, in a suitable solvent to form an imine intermediate, which is subjected to reducing conditions to form the product represented by structural formula IV. Such reactions are well-known in the art; see, for example, Larock, pp 835-839.

In the reductive amination, the aldehyde III, an imine reducing agent, and an optional acid are each used in a molar ratio independently selected from about 1 to about 10 relative to nojirimycin V, and are used with a polar solvent, a polar protic solvent, a halogenated solvent, an ethereal solvent, or an aromatic solvent. The reagents can be added in any order, or all at once. Preferably, aldehyde III, the reducing agent, and the optional acid are each used in a molar ratio independently selected from about 1 to about 5 relative to nojirimycin V, and the solvent is a polar protic solvent, a halogenated solvent, or an ethereal solvent. More preferably, the aldehyde III, the reducing agent, and the optional acid are each used in a molar ratio independently selected from about 1 to about 2 relative to nojirimycin V, and the solvent is a polar protic solvent, e.g., an alcohol, preferably ethanol. In one example, the aldehyde III and NaCNBH₃ are each in a molar ratio of about 1.5 and glacial acetic acid is used in a molar ratio of about 1 relative to nojirimycin V, and the solvent used is ethanol. The reaction temperature is in a range from about 0° C. to about 40° C., preferably about ambient temperature. The reagents can be added in any order or simultaneously, preferably simultaneously. Representative conditions are provided in Example 1 in the Exemplification.

Reductive amination reactions require an imine reducing agent, i.e., a reducing reagent which can convert and imine to an amine, for example, electrolytic reduction or a reagent such as a borohydride reducing reagent, e.g., NaCNBH₃, BH₃, NaBH4, NaCNBH4, Na(CH₃CO2)₃BH, and the like, or a hydride reducing reagent, e.g., LiAlH₃, Zn, H₂-Raney nickel, H₂—Pt, H₂—Pd, and the like. In the present invention, borohydride reagents, e.g., NaCNBH4 or Na(CH₃CO₂)₃BH, are commonly used. Reductive amination reactions optionally include an acid such as HCl, HI, HBr, glacial acetic acid, and the like, preferably glacial acetic acid. The reducing agent can incorporate the optional acid; for example, Na(CH₃CO₂)₃BH contains the acetate group as a ligand.

Suitable reaction temperatures are in the liquid range of the reaction solvent, for example, between ambient temperature (about 15°-25° C.) and the boiling point of the solvent, or between and ambient temperature and the freezing point of the solvent. The choice of temperature depends on the rate of each reaction and the stability of the reaction products. For example, when a reaction is strongly exothermic, and/or the solvent is not liquid at ambient temperature, the reaction is run at a temperature in the liquid range of the solvent and below room temperature, e.g., between the freezing point of the solvent and ambient temperature, typically between −78° to 10° C., or preferably between −78° and 0° C. For example, in a lithium reduction carried out in liquid ammonia (Example 3), the temperature is between −78° to −30° C., or preferably about −78° C. Alternatively, reactions that proceed slowly at ambient temperature can be run at higher temperatures to increase the reaction rate, up to the boiling point of the solvent, provided that the products do not significantly decompose under those conditions. For example, in Example 6, adamantyl methanol and 5-bromo-1-pentene are reacted in dimethyl sulfoxide at 70° C. One skilled in the art will recognize that reactions may need to be heated or cooled to maintain a preferred reaction temperature, and in particular, reactions that are only mildly exothermic and require no cooling on a laboratory scale may require significant cooling when scaled up, for example, in production.

A suitable solvent can be any solvent in which at least one, and preferably all of the reagents and products are soluble and which does not interfere with the course of the reaction or react with the reagents. Depending on the reaction, suitable solvents can include polar protic solvents such as water, ethanol, 2-propanol, and ethylene glycol; ethereal solvents such as diethyl ether and tetrahydrofuran; polar aprotic solvents such as dimethyl sulfoxide, dimethyl formamide, and N-methylpyrrolidone; halogenated solvents such as chloroform, carbon tetrachloride, methylene chloride, chloroform, and 1,2 dichloroethylene; aromatic solvents such as benzene, toluene, nitrobenzene, and xylene; and the like.

For example, in the reductive amination, one skilled in the art will know to choose suitable solvents based on the various reagents. For example, when R3 is —H, as described above, a preferred solvent is a polar protic solvent, e.g., an alcohol, for example, ethanol, methanol, or 2-propanol, most preferably, ethanol. In alternatives where R3 is a protecting group, i.e., is less polar than when R3 is —H, the solvent is preferably relatively nonpolar, for example, an aromatic, halogenated, or ethereal solvent, or more preferably, benzene or 1,2-dichlorethane, or most preferably, 1,2-dichloroethane.

Suitable protecting groups represented by R3 include alcohol protecting groups, for example, methyl, methoxymethyl, trimethylsilyl, tert-butyl, benzyl, and the like. The use of protecting groups is well-known in the art, as described extensively in Chapter 2 of Greene, T W; Wuts, P G M; “Protective Groups in Organic Synthesis,” 3^{rd} Ed, 1991, Wiley & Sons, New York, and references cited therein, the entire teachings of which are incorporated herein by reference.

For example, benzyl protected nojirimycin represented by structural formula Vb is commonly used and can be prepared from commercially available 2,3,4,6-tetra-O-benzyl-D-galactopyranose (Pfanstiehl, Waukegan, Ill.) according to Matos, C R R; Lopes, R S C; Lopes, C C. Synthesis, 1999, 4, 571-572, the entire teachings of which are incorporated herein by reference.

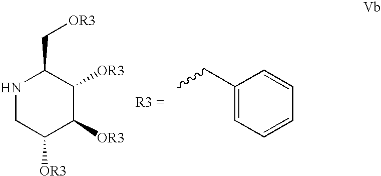

For example, in one alternative reductive amination, the acid is omitted, the reducing agent is a borohydride reducing reagent, e.g., Na(CH₃CO₂)₃BH, the aldehyde is represented by structural formula II, the substrate is represented by structural formula Vb, and the solvent used is an aromatic or halogenated solvent. Relative to Vb, the Na(CH₃CO₂)₃BH and aldehyde II can each be used in a molar ratio between about 1 to about 10, or alternatively, between about 1 to about 5. Preferably, relative to Vb, the aldehyde represented by structural formula II is in a molar ratio of about 1.5, Na(CH₃CO₂)₃BH is in a molar ratio of about 4, and the solvent is 1,2 dichloroethylene. The reagents can be added in any order, simultaneously, or the reducing agent is added last after all the other reagents have been combined with the solvent. Preferably, the reducing agent is added last. The reaction temperature can be any temperature in the liquid range of the solvent, preferably about ambient temperature. Representative conditions are provided in Example 2 in the Exemplification.

When R3 is a protecting group, an additional deprotection step is included. Such deprotection steps are also referenced extensively in “Protective Groups in Organic Synthesis”, above. For example, if, as above, R3 is benzyl, resulting in a tetrabenzyl intermediate, a reductive cleavage step can be used. A reductive cleavage step employs a reducing agent, for example, electrolytic reduction or an excess of reagents such as H₂ with a Pd catalyst, alkali metal in liquid ammonia, and the like. Preferably, a reductive cleavage step comprises a large excess of lithium dissolved in liquid ammonia, i.e., lithium in a molar ratio relative to each protecting group of between about 5 to about 50, alternatively 25 to about 50, and preferably about 35. The reaction is run at reduced temperature, i.e., between about −78° C. and −30° C., preferably at about −78° C. Representative conditions are provided in Example 3 in the Exemplification.

R1 is an optionally substituted aliphatic group or an optionally substituted aryl group, for example, a polycyclic alkane, a polycyclic aryl group, an alkyl chain, and the like. Preferably, R1 is an optionally substituted group represented by one of structural formulas i-v. 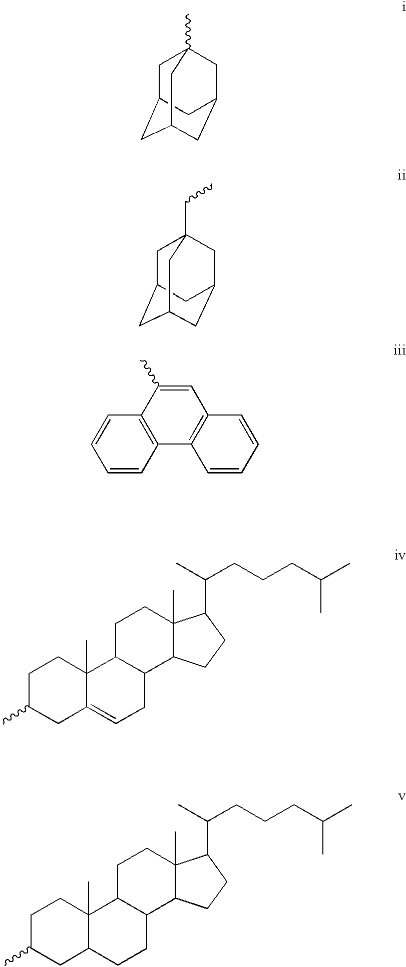
Preferably, suitable optional substituents for R1 include —OH, —CN, —NO₂, —R^{a}, —OR^{a}, —COR^{a}, —CO₂R^{a}, —NR^{a}₂, halogen, or optionally substituted aryl, heteroaryl, aralkyl, or heteroaralkyl groups, wherein R^{a} is a C₁-C₂₆ branched or linear aliphatic group. Alternatively, R1 is an unsubstituted group represented by one of structural formulas i-v. More preferably, R1 is a group represented by structural formulas ii or iv. Most preferably, R1 is represented by structural formula ii.

R2 is an aliphatic linking group, for example, an alkyl chain that contains zero, one, or more units of unsaturation, a cycloalkane ring, and the like. Alternatively, R2 is —(CH₂)ₙ— and n is 1 to 6. More preferably, n is 1 to 4. Most preferably, n is 3.

In a preferred embodiment, R1 is a group represented by one of structural formulas i-v and R2 is an alkyl chain. Alternatively, R2 is —(CH₂)ₙ— and n is 1 to 6. More preferably, n is 1 to 4. Most preferably, n is 3.

In a more preferred embodiment, R1 is a group represented by structural formulas ii or iv, R2 is —(CH₂)ₙ— and n is 1 to 6. More preferably, n is 1 to 4. Most preferably, n is 3.

Even more preferably, R1 is represented by structural formulas ii or iv and R2 is —(CH₂)₃—.

As used herein, an aliphatic linking group is any group that connects two other groups and does not substantially interfere with the reactions described herein, or with the pharmacological activity of the final product. A linking group can be, for example, an alkyl chain, an aliphatic chain, a cycloalkyl ring, and the like. “Interfering with a reaction” refers to substantially decreasing the yield (e.g., a decrease of greater than 50%) or causing a substantial amount of by-product formation (e.g., where by-products represent at least 50% of the theoretical yield). Interfering substituents can be used, provided that they are first converted to a protected form. Suitable protecting groups are known in the art and are disclosed, for example, in “Protective Groups in Organic Synthesis”, above.

As used herein, an aliphatic group is a straight chained, branched, cyclic, polycyclic, or bridged (non-aromatic) hydrocarbon which is completely saturated or which contains one or more units of unsaturation. Typically, a straight chained or branched aliphatic group has from one to about twenty six carbon atoms, preferably from one to about ten, and a cyclic aliphatic group has from three to about eight ring carbon atoms per ring. An aliphatic group is preferably a completely saturated, straight-chained or branched alkyl group, e.g., methyl, ethyl, n-propyl, 2-propyl, n-butyl, sec-butyl, tert-butyl, pentyl, hexyl, heptyl or octyl, or a cycloalkyl group with three to about eight ring carbon atoms per ring. Other aliphatic groups include polycyclic groups such as adamantyl, adamantly methyl, cholesterol, cholestenol, bicyclo[2.2.2]octane, and the like. Aliphatic groups may additionally be substituted or be interrupted by another group.

Aryl groups include carbocyclic aromatic groups such as phenyl, naphthyl, and anthracyl, and heteroaryl groups such as imidazolyl, isoimidazolyl, thienyl, furanyl, pyridyl, pyrimidyl, pyranyl, pyrrolyl, pyrazolyl, pyrazinyl, thiazolyl, isothiazolyl, oxazolyl, isooxazolyl, 1,2,3-triazolyl, 1,2,4-triazolyl, and tetrazolyl.

Aryl groups also include fused polycyclic aromatic ring systems in which a carbocyclic aromatic ring or heteroaryl ring is fused to one or more other heteroaryl rings. Examples include benzothienyl, benzofuranyl, indolyl, isoindolyl, quinolinyl, benzothiazolyl, benzoisothiazolyl, benzooxazolyl, benzoisooxazolyl, benzimidazolyl, indolizinyl, quinolinyl, and isoquinolinyl.

Suitable substituents on alkyl, aliphatic, aryl, or non-aromatic heterocyclic groups, for example, the alkyl, aliphatic, and aryl groups represented by R1, are those that do not substantially interfere with the reactions described herein, and provide pharmaceutically active compounds. Suitable substituents on an alkyl, aliphatic, aryl, or non-aromatic heterocyclic groups include, for example, —OH, halogen (—Br, —Cl, —I and —F); optionally substituted aryl, heteroaryl, aralkyl, or heteroaralkyl groups; —OR^{b}, —O—COR^{b}, —COR^{b}, —CN, —NO₂, —COOH, —SO₃H, —NH₂, —NHR^{b}, —N(R^{b}R^{c}), —COOR^{b}, —CHO, —CONH₂, —CONHR^{b}, —CON(R^{b}R^{c}), —NHCOR^{b}, NRCCOR^{b}, —NHCONH₂, —NHCONR^{b}H, —NHCON(R^{b}R^{c}), —NR^{d}CONH₂, —NR^{d}CONR^{b}H, —NR^{d}CON(R^{b}R^{c}), —NHNH₂, —SO₂NH₂, —SO₂NHR^{b}, —SO₂NR^{b}R^{c}, —SH, and —NH—C(═NH)—NH₂. R^{b}—R^{e} each are independently an optionally substituted aliphatic, aryl, aralkyl, heteroaryl, or heteroaralkyl, preferably an alkyl, benzylic or aryl group. In addition, —NR^{b}R^{c}, taken together, can also form a substituted or unsubstituted non-aromatic heterocyclic group.

### EXEMPLIFICATION

The present invention is illustrated by the following examples, which are not intended to be limiting in any way. The other compounds disclosed herein can be prepared by modifying the following methods with suitable choice of starting materials.

### Example 1

### Improved Coupling of 1-Deoxynojirimycin and Aldehyde 2

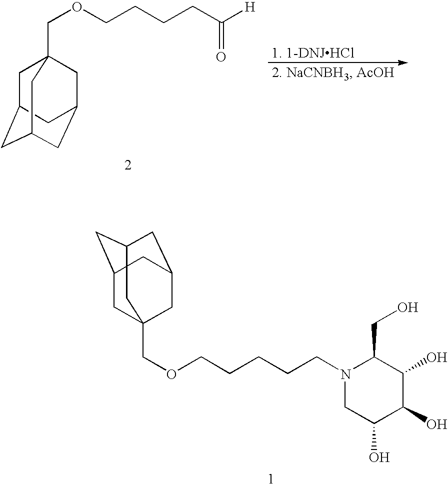

A reaction flask was charged with 2 (3.20 g, 12.8 mmol), ethanol (100 mL), 1-deoxynojirimycin hydrochloride (1-DNJ.HCl, 1.70 g, 8.53 mmol), glacial acetic acid (AcOH, 0.5 mL) and NaCNBH₃ (804 mg, 12.8 mmol), under dry N₂. After stirring for 48 h, the mixture had become a milky white suspension. The solvent was removed and the off-white glassy residue was taken up in 50 mL of 5% HCl, resulting in gas evolution. The resulting soapy mixture was neutralized to pH 7.5 using solid Na₂CO₃ and then extracted with CHCl₃. The combined extracts were passed through celite and the solvent removed to afford an off-white glassy solid. Purification on a 350 g silica gel column, using a solvent mixture of 700 mL CHCl₃, 200 mL methanol, and 20 mL of 28% NH₄OH, followed by solvent removal gave 1 (2.41 g, 0.606 mmol) in 74% yield.

### Example 2

### Coupling of Tetrabenzyl 1-Deoxynojirimycin and Aldehyde 2

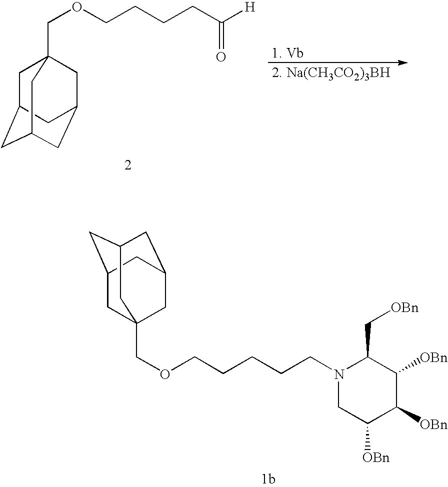

A flask was charged with 2 (143 mg, 0.573 mmol), tetrabenzyl-1-deoxynojirimycin (Vb, 200 mg, 0.382 mmol, prepared from 2,3,4,6-tetra-O-benzyl-D-galactopyranose (Pfanstiehl, Waukegan, Ill.) according to Matos, C R R; Lopes, R S C; Lopes, C C. Synthesis, 1999, 4, 571-572,) and 1,2 dichlorethylene (6 mL), and the mixture was allowed to stir for 15 min, at which time Na(CH₃CO₂)₃BH (0.324 mg, 1.53 mmol) was added in one portion. The resulting mixture was stirred for 3 h. Subsequently, 25 mL of sat aq NaHCO₃ and 25 mL of CHCl₃ were added. The organic layer was dried and the solvent removed. The resulting orange oil was purified on a silica gel column using 10% ethyl acetate in hexane as the solvent to afford compound 1b (170 mg, 224 mmol) in 59% yield.

### Example 3

### Deprotection of Tetrabenzyl Compound 1b to form Compound 1

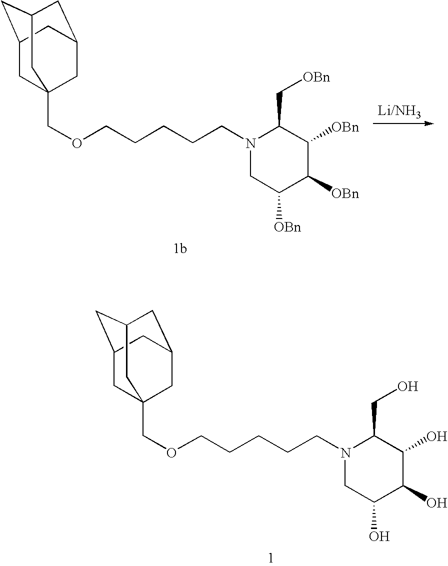

A dry flask was charged with Li (196 mg, 28.2 mmol) under dry N₂, cooled to −78° C. and 30 mL of liquid NH₃ was condensed into the flask. The mixture was stirred until the Li dissolved in the NH₃ to afford a blue solution. Subsequently, a solution of compound 1b (150 mg, 0.198 mmol) in dry tetrahydrofuran was added via syringe. After 10 min, an additional amount of Li (196 mg, 28.2 mmol) was added. After 3 h, the mixture was allowed to rise to room temperature and the NH₃ was allowed to evaporate under a stream of N₂. The residue was combined with 20 mL of H₂O, followed by addition of an ion exchange resin (10 g, Dowex 50W-X8 100) and the mixture was stirred for 2 h. The resin was filtered and washed with 1 N NH₄OH (20 mL) and 7 N NH₃ in methanol. The combined aqueous fractions were extracted with ethyl acetate. Drying and solvent removal of the organic extracts afforded compound 1 (43 mg, 108 mmol) in 55% yield as an orange oil.

### Example 4

### Improved Synthesis of Aldehyde 2

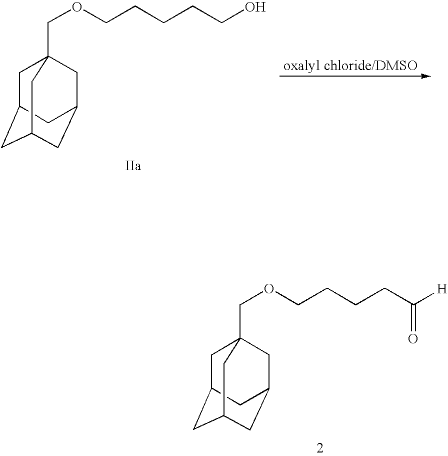

A dry flask was charged with anhydrous CH₂Cl₂ (25 mL) and oxalyl chloride (14.1 mL, 28.2 mmol) under anhydrous N₂. The mixture was cooled to −70° C. and a solution of dimethyl sulfoxide (DMSO, 3.09 mL, 43.5 mmol) in anhydrous CH₂Cl₂ (5 mL) was added in a dropwise manner. After 5 min, compound 1a (3.23 g, 12.8 mmol) was added and the mixture was stirred for 15 min, becoming a milky yellow. Triethylamine (8.87 mL, 64.0 mmol) was added and the mixture stirred for 5 min, then allowed to warm to room temperature, followed by addition of H₂O (50 mL) and extraction with CH₂Cl₂. Drying and solvent removal from the combined extracts afforded compound 2 (3.04 g, 12.2 mmol) in 95% yield as a clear, colorless oil.

### Example 5

### Improved Synthesis of Alcohol IIa

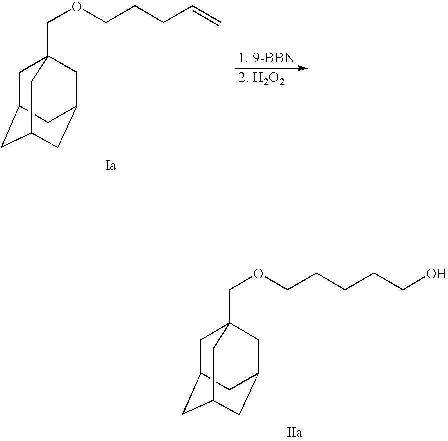

A solution of alkene Ia (3.37 g, 14.4 mmol) in diethyl ether was added to a flask under anhydrous N₂ and then cooled in an ice bath. Tetrahydrofuran (75 ml) was added to the mixture. Subsequently, 9-borobicyclo[3.3.1]nonane (9-BBN, 43.1 mL, 21.6 mmol) was added in a dropwise manner. The resulting mixture was stirred and allowed to rise to room temperature overnight. The mixture was cooled again with an ice bath and 1N aq NaOH (86 mL 86 mmol) was added with stirring, followed 10 min later by a portion of 30% H₂O₂ (7 mL). The solution became warm and was allowed to stir for 3 h, and the mixture was extracted with diethyl ether. Drying of the organic extracts followed by solvent removal gave compound 1a (3.23 g, 12.8 mmol) in 89% yield as a clear, colorless oil.

### Example 6

### Improved Synthesis of Alkene Ia

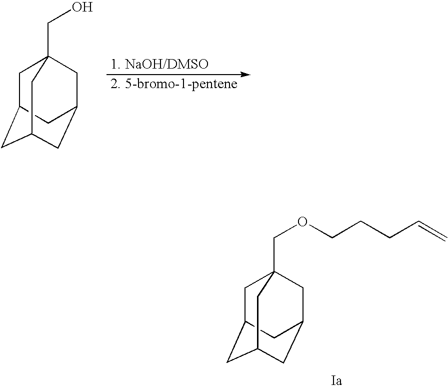

Adamantyl methanol (13.9 g, 83.9 mmol) and 5-bromo-1-pentene (25.0 g, 168 mmol) were added to a suspension of pulverized KOH (18.8 g, 336 mmol) in 275 mL of DMSO. The mixture was stirred and heated to 70° C. overnight. The resulting yellow mixture was poured into 200 mL of H₂O, extracted with diethyl ether, and the combined ether extracts evaporated to form a wet crystalline residue. The residue was redissolved in ether and passed through a silica plug to remove unreacted starting material. Drying of the organic extracts followed by solvent removal gave compound 1a (12.85 g, 54.9 mmol) in 65.4% yield as a clear, colorless oil.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of preparing a compound represented by structural formula IV: 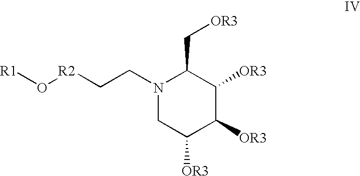
comprising reacting a compound represented by structural formula II with an oxidizing agent: 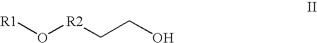
thereby forming a compound represented by structural formula III: 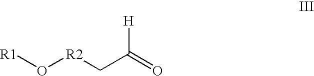
and reductively aminating the compound represented by structural formula III with a 1-deoxynojirimycin derivative represented by structural formula V: 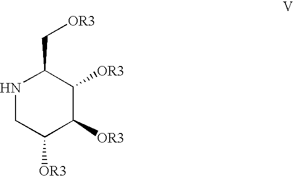
thereby forming the compound represented by structural formula IV, wherein:
R1 is an optionally substituted aliphatic or aryl group;
R2 is an optionally substituted aliphatic linking group; and
each R3 is independently —H or an alcohol protecting group.

2. The method of claim 1, wherein R3 is an alcohol protecting group, further comprising deprotecting the alcohol groups to form a compound represented by structural formula IVa:

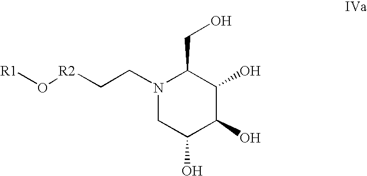

3. The method of claim 2, wherein R3 is a benzyl group, and wherein the deprotection step comprises reaction of the compound represented by structural formula IVa with lithium dissolved in ammonia.

4. The method of claim 1, wherein the compound represented by structural formula II is prepared by hydroborating a compound represented by structural formula I: 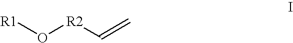
thereby forming a hydroborated intermediate, and oxidizing the intermediate to form the compound represented by structural formula II.

5. The method of claim 4, further comprising the step of preparing the compound represented by structural formula I by alkylating a compound represented by structural formula R1-OH with a compound represented by structural formula Y—R2—CH═CH₂, wherein Y is a leaving group.

6. The method of claim 5, wherein Y is —Br.

7. The method of claim 1, wherein R1 is a group represented by a structural formula selected from i, ii, iii, iv, and v:

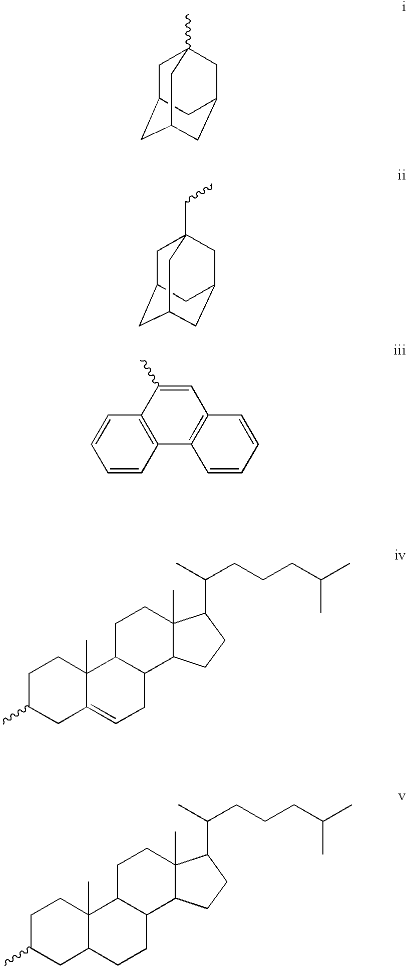

8. The method of claim 7, wherein R1 is a group represented by one of structural formulas ii or iv.

9. The method of claim 1, wherein R2 is —(CH₂)ₙ— and n is 1 to 6.

10. The method of claim 9, wherein R3 is —H.

11. A method of preparing a compound represented by structural formula III, 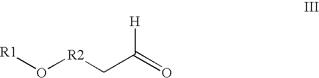
comprising the step of oxidizing a compound represented by structural formula II: 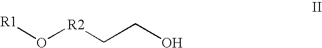
wherein:
R1 is a group represented by a structural formula selected from i, ii, iii, iv, and v; 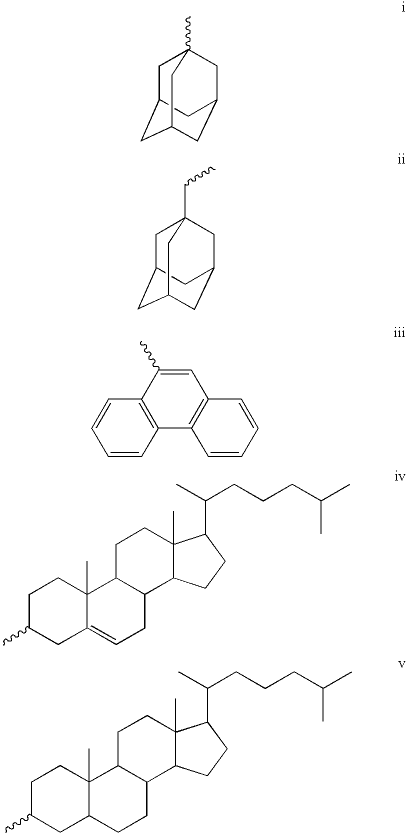
wherein R1 is optionally substituted; and
R2 is an aliphatic linking group.

12. The method of claim 11, wherein the compound represented by structural formula II is prepared by hydroborating a compound represented by structural formula I: 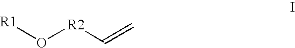
thereby forming a hydroborated intermediate, and oxidizing the intermediate,
thereby forming the compound represented by structural formula II.

13. The method of claim 12, further comprising the step of preparing the compound represented by structural formula I by reacting a compound represented by structural formula R1-OH with a compound represented by structural formula Y—R2—CH═CH₂, wherein Y is a leaving group.

14. The method of claim 11, wherein the compound represented by structural formula II is oxidized with an oxidizing agent selected from the group consisting of a mixture of pyridine with CrO₃, pyridinium chlorochromate, potassium dichromate, sodium dichromate, and a mixture of oxalyl chloride with dimethyl sulfoxide.

15. A method of preparing a compound represented by structural formula II: 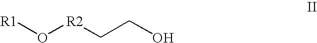
by hydroborating a compound represented by structural formula I: 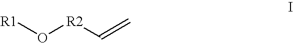
thereby forming a hydroborated intermediate, followed by oxidizing the intermediate to form the compound represented by structural formula II; wherein:
R1 is a group represented by a structural formula selected from i, ii, iii, iv, and v; 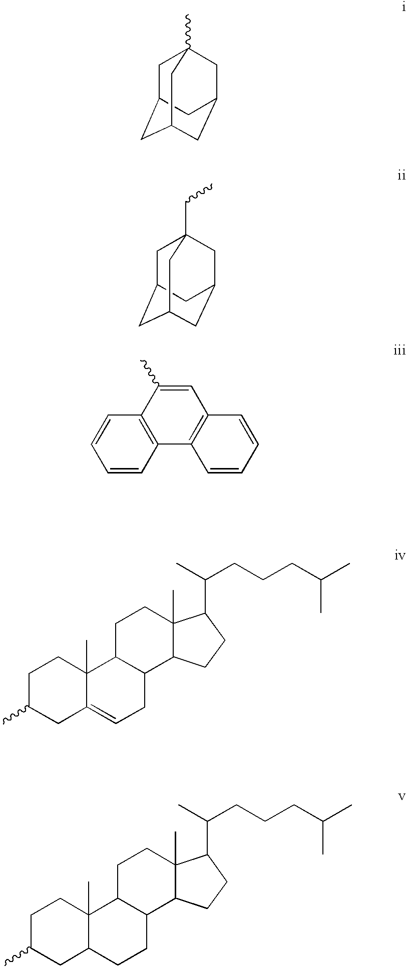
wherein R1 is optionally substituted; and
R2 is an aliphatic linking group.

16. The method of claim 15, further comprising the step of preparing the compound represented by structural formula I by reacting a compound represented by structural formula R1—OH with a compound represented by structural formula Y—R2—CH═CH₂, wherein Y is a leaving group.

17. The method of claim 15, wherein:
a. the compound represented by structural formula II is hydroborated with a reagent selected from the group consisting of BH₃, B₂H₆, bis(3-methyl-2-butyl)borane, BH₂Cl, and 9-borobicyclo[3.3.1]nonane; and
b. the hydroborated intermediate is oxidized with H₂O₂.

18. A method of preparing a compound represented by structural formula I, 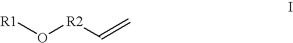
by reacting a compound represented by structural formula R1-OH with a compound represented by structural formula Y—R2-CH═CH₂, wherein:
Y is a leaving group;
R1 is a group represented by a structural formula selected from i, ii, iii, iv, and v; 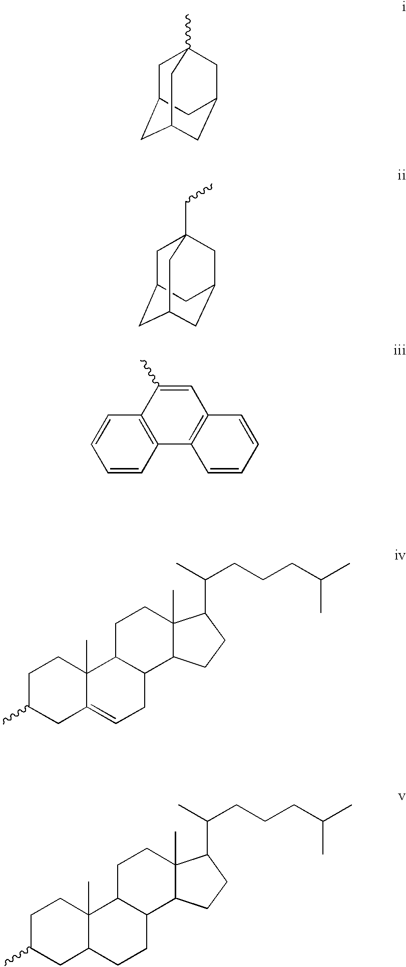
wherein R1 is optionally substituted; and
R2 is an aliphatic linking group.

19. A compound represented by structural formula VI, 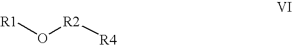
wherein
R1 is a group represented by a structural formula selected from i, ii, iii, iv, and v; 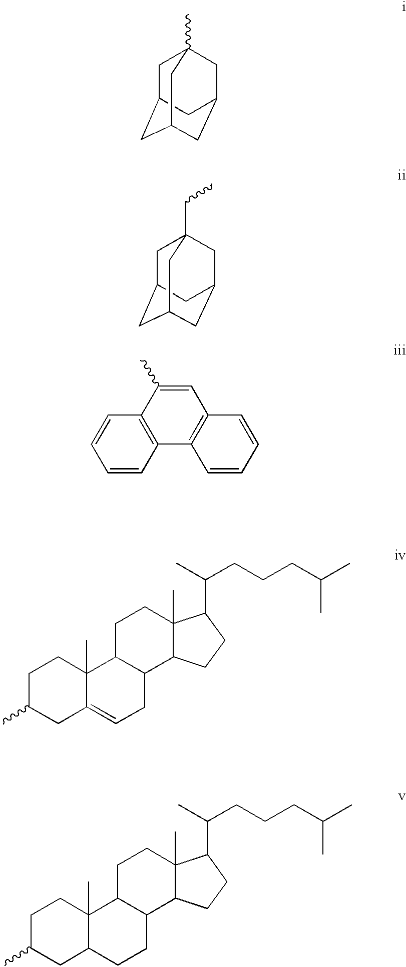
wherein R1 is optionally substituted;
R2 is an aliphatic linking group; and
R4 is —CH═CH₂ or —CH₂CH₂OH.

20. A method of preparing a compound represented by structural formula IVa: 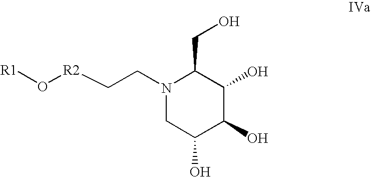
comprising
a) alkylating a compound represented by structural formula R1—OH with a compound represented by structural formula Br—R2—CH═CH₂, thereby forming a compound represented by structural formula I: 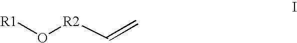
b) hydroborating the compound represented by structural formula I with 9-borobicyclo[3.3.1]nonane, thereby forming a hydroborated intermediate, and oxidizing the intermediate with H₂O₂, thereby forming a compound represented by structural formula II: 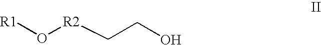
c) reacting the compound represented by structural formula II with a mixture of oxalyl chloride with dimethyl sulfoxide, thereby forming a compound represented by structural formula III: 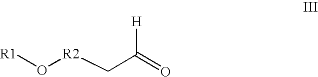
d) reductively aminating the compound represented by structural formula III with 1-deoxynojirimycin represented by structural formula Va: 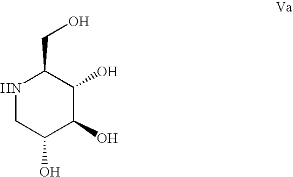
thereby forming the compound represented by structural formula IVa; wherein:
R1 is a group represented by a structural formula selected from i, ii, iii, iv, and v: 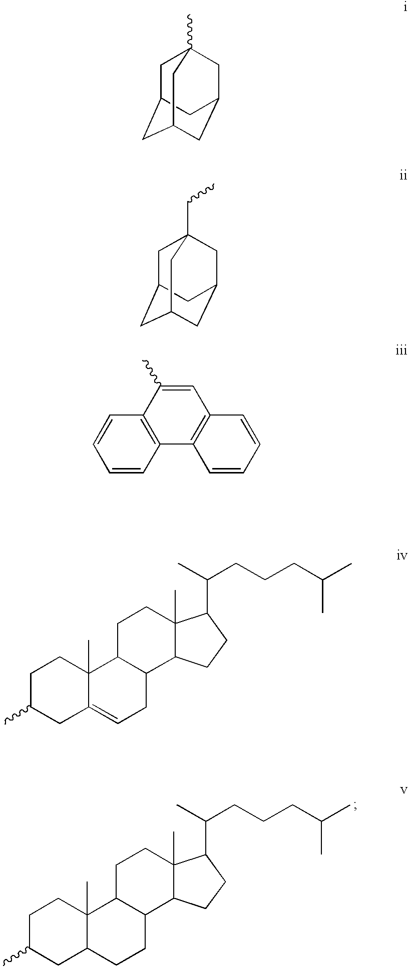
and
R2 is —(CH₂)₃—.
